(19) United States
(12) Patent Application Publication
(10) Pub. No.: US 2022/0362107 A1
(43) Pub. Date: Nov. 17, 2022

(54) METHOD FOR PREPARING ANTI-HEPATOMA DRUG SANJIE TABLET

(71) Applicant: ZHUHAI MED-TECH BIOPHARM CO., LTD., ZHUHAI (CN)

(72) Inventors: Shanshan GUO, Shenzhen (CN); Dong WANG, Shenyang (CN); Xiukun WU, Shenzhen (CN); Liang XU, Xi'an (CN); Xuewu CHEN, Leizhou (CN); Jun LIU, Shenyang (CN)

(21) Appl. No.: 17/447,179

(22) Filed: Sep. 8, 2021

(30) Foreign Application Priority Data
May 15, 2021 (CN) 202110529988.0

(51) Int. Cl. A61J 3/00; A61K 36/888; A61K 36/52; A61K 36/75; A61K 35/413; A61K 35/745; A61K 36/064; A61K 35/747

(52) U.S. Cl. CPC A61J 3/005; A61K 36/888; A61K 36/52; A61K 36/75; A61K 35/413; A61K 35/745; A61K 36/064; A61K 35/747

(57) ABSTRACT

The present disclosure discloses a method for preparing an anti-hepatoma drug sanjie tablet, which includes a sanjie tablet containing components of Typhonii Rhizoma, exocarp of Juglans mandshurica, Dictamni Cortex and Bovis Calculus Artifactus. The preparation method includes the steps of: step 1. removing toxins from the components of Typhonii Rhizoma, exocarp of Juglans mandshurica and Dictamni Cortex in the sanjie tablet to obtain powder of nontoxic Typhonii Rhizoma, nontoxic exocarp of Juglans mandshurica, and nontoxic Dictamni Cortex; and step 2, uniformly mixing the powder of nontoxic Typhonii Rhizoma, nontoxic exocarp of Juglans mandshurica, and nontoxic Dictamni Cortex obtained in step 1 with Bovis Calculus Artifactus, granulating with a volatile medium, drying, tabletting, and conducting film-coating to obtain the sanjie tablet. Beneficial effects: the components in the composition of the sanjie tablet is decomposed and transformed by probiotics, and improving the medication safety of a patient in clinic.

### CROSS-REFERENCE TO RELATED APPLICATIONS

This is a U. S. patent application which claims the priority and benefit of Chinese Patent Application Number 202110529988.0, filed on May 15, 2021, the disclosure of which is incorporated herein by reference in its entirety.

### TECHNICAL FIELD

The present disclosure relates to the technical field of biomedicine, and in particular to a method for preparing an anti-hepatoma drug sanjie tablet.

### BACKGROUND

The Chinese invention patent with the application number of CN200410050749.3 discloses a traditional Chinese medicine for treating hepatoma and a preparation process thereof, wherein the preparation process is that: Typhonii Rhizoma, Dictamni Cortex and exocarp of Juglans mandshurica are taken according to the weight ratio in the raw medical materials and added with water for decocting twice, wherein the amount of the water added for the first time is 12 times of the total weight of the raw medical materials, decocting is conducted for 2 hours, and the amount of the water added for the second time is 8 times of the total weight of the raw medical materials, and decocting is conducted for 1 hour; then filtering is conducted, and filtrates are combined and concentrated into a concentrated cream with the relative density of 1.35-1.38 and the temperature of 70° C., dried under reduced pressure, crushed, added with the fine powder of Bovis Calculus Artifactus and auxiliary materials, mixed uniformly, granulated with 75% ethanol, dried, tabletted, and coated with a thin film to obtain the sanjie tablet. The disadvantage of this method lies in that the toxins in exocarp of Juglans mandshurica and Typhonii Rhizoma cannot be removed, so that the toxins in the exocarp of Juglans mandshurica and Typhonii Rhizoma are made into the sanjie tablet together with exocarp of Juglans mandshurica and Typhonii Rhizoma, thereby reducing the pharmaceutical effect of the sanjie tablet.

### SUMMARY

With the main objective of overcoming the aforementioned technical problems, the present disclosure provides a method for preparing an anti-hepatoma drug sanjie tablet, which includes the following steps: firstly, removing toxins contained in the components of the sanjie tablet, and then preparing the components from which the toxins have been removed into the sanjie tablet for improving the pharmaceutical effect.

In order to overcome the aforementioned technical problems, the technical solution adopted by the present disclosure is as follows.

Provided is a method for preparing an anti-hepatoma drug sanjie tablet, which includes a sanjie tablet containing components of Typhonii Rhizoma, exocarp of Juglans mandshurica, Dictamni Cortex and Bovis Calculus Artifactus, the preparation method including the steps of:

step 1. removing toxins from the components of Typhonii Rhizoma, exocarp of Juglans mandshurica, and Dictamni Cortex in the sanjie tablet to obtain powder of nontoxic Typhonii Rhizoma, nontoxic exocarp of Juglans mandshurica, and nontoxic Dictamni Cortex; and

step 2. uniformly mixing the powder of nontoxic Typhonii Rhizoma, nontoxic exocarp of Juglans mandshurica, and nontoxic Dictamni Cortex obtained in step 1 with Bovis Calculus Artifactus, granulating with a volatile medium, drying, tabletting, and conducting film-coating to obtain the sanjie tablet. The pharmaceutical effect of the sanjie tablet is improved.

Further, the removing toxins from the exocarp of Juglans mandshurica component in the sanjie tablet includes the steps of:

step 1. directly preparing fresh exocarp of Juglans mandshurica into a homogenate of exocarp of Juglans mandshurica, or drying and crushing fresh exocarp of Juglans mandshurica into coarse powder of exocarp of Juglans mandshurica;

step 2. taking probiotics and evenly mixing the same with distilled water at 20-40° C. according to the mass ratio of 1:5-50 to prepare a probiotic solution;

step 3. weighing 1 kg of the homogenate or coarse powder of exocarp of Juglans mandshurica from step 1, weighing 2-10 L of the probiotic solution from step 2, and mixing 1 kg of the homogenate or coarse powder of exocarp of Juglans mandshurica with 2-10 L of the probiotic solution according to a certain ratio to obtain a mixed raw stock slurry;

step 4. fermenting and culturing the mixed raw stock slurry obtained in the step 3 at a fermentable conversion temperature for 1 cycle, so as to conduct fermentable conversion with a ventilation ratio of 0-10 v/v and a stirring rate of 0-300 r/min to obtain a fermentation mixture; and

step 5. freeze-drying or oven-drying the fermentation mixture obtained in step 4 at a temperature of 40-80° C., and then crushing into fine powder as the nontoxic powder of exocarp of Juglans mandshurica.

Further, the probiotics is any one of probiotic Bacillus, clostridium butyicum, lactobacillus, bifidobacterium, saccharomycetes, medicated leaven and red yeast rice, or is at least prepared from any two or more of probiotic Bacillus, Clostridium butyicum, lactobacillus, bifidobacterium, saccharomycetes, medicated leaven and red yeast rice.

Further, the OD600 of the probiotic solution >0.4.

Further, the fermentable conversion temperature is 25-45° C., and 1 fermentation cycle is 1-14 days.

The removing toxins from the Typhonii Rhizoma component in the sanjie tablet includes the steps of:

step 1. crushing crude Typhonii Rhizoma into coarse powder;

step 2. taking probiotics and evenly mixing the same with distilled water at 20-40° C. according to the mass ratio of 1:5-50 to prepare a probiotic solution;

step 3. weighing 1 kg of crude Typhonii Rhizoma from step 2 and crushing into coarse powder, weighing 2-10 L of the probiotic solution from step 2, and mixing 1 kg of the coarse powder of crude Typhonii Rhizoma with 2-10 L of the probiotic solution according to a certain ratio to obtain a mixed proportional slurry;

step 4. fermenting and culturing the mixed proportional raw stock slurry obtained in step 3 at a fermentable conversion temperature for 1 cycle, so as to conduct fermentable conversion with a ventilation ratio of 0-10 v/v and a stirring rate of 0-300 r/min to obtain a proportional fermentation mixture; and

step 5. freeze-drying or oven-drying the proportional fermentation mixture obtained in step 4 at a temperature of 40-80° C., and then crushing into fine powder as the nontoxic powder of Typhonii Rhizoma.

Further, the probiotics is any one of probiotic Bacillus, Clostridium butyicum, lactobacillus, bifidobacterium, saccharomycetes, medicated leaven and red yeast rice, or is at least prepared from any two or more of probiotic Bacillus, Clostridium butyicum, lactobacillus, bifidobacterium, saccharomycetes, medicated leaven and red yeast rice.

Further, the OD600 of the probiotic solution >0.4.

Further, the fermentable conversion temperature is 25-45° C., and 1 fermentation cycle is 1-14 days.

Further, the removing toxins from the Dictamni Cortex component in the sanjie tablet includes the steps of:

step 1. crushing Dictamni Cortex into coarse powder of cortex dictamni;

step 2. taking probiotics and evenly mixing the same with distilled water at 20-40° C. according to the mass ratio of 1:5-50 to prepare a probiotic solution;

step 3. weighing 1 kg of the coarse powder of Dictamni Cortex from step 1, weighing 2-10 L of the probiotic solution from step 2, and mixing 1 kg of the coarse powder of Dictamni Cortex with 2-10 L of the probiotic solution according to a certain ratio to obtain a mixed raw stock slurry;

step 4. fermenting and culturing the mixed raw stock slurry obtained in the step 3 at a fermentable conversion temperature for 1 cycle, so as to conduct fermentable conversion with a ventilation ratio of 0-10 v/v and a stirring rate of 0-300 r/min to obtain a fermentation mixture; and

step 5. freeze-drying or oven-drying the fermentation mixture obtained in step 4 at a temperature of 40-80° C., and then crushing into fine powder as the nontoxic powder of cortex dictamni.

Further, the probiotics is any one of probiotic Bacillus, Clostridium butyicum, lactobacillus, bifidobacterium, saccharomycetes, medicated leaven and red yeast rice, or is at least prepared from any two or more of probiotic Bacillus, Clostridium butyicum, lactobacillus, bifidobacterium, saccharomycetes, medicated leaven and red yeast rice.

Further, the OD600 of the probiotic solution >0.4.

Further, the fermentable conversion temperature is 25-45° C., and 1 fermentation cycle is 1-14 days.

Further, the volatile medium is ethanol.

Compared with the prior art, the present disclosure has the following beneficial effects.

The components in the composition of the sanjie tablet is decomposed and transformed by probiotics, thereby thoroughly removing toxic components of the traditional Chinese medicine, greatly improving the pharmaceutical effect and the absorption effect, and improving the medication safety of a patient in clinic.

The sanjie tablet obtained by the preparation method of the present disclosure has the following advantages.

1. The anti-tumor active components in the components of the sanjie tablet is converted from macromolecules to micromolecules, and the effective components can quickly penetrate the walls of blood capillaries to reach the tumor focus directly.

2. The undeveloped anti-tumor active substance factors of the sanjie tablet are activated, and differentiation and apoptosis of the tumor cells are induced.

3. The functions of multi-target targeted recognizing, inhibiting and killing tumor cells, inhibiting tumor neovascularization, preventing recurrence and metastasis of the sanjie tablet are achieved, and the pharmaceutical effect is improved by 4 to 28 times.

4. The therapeutic effect of the sanjie tablet on digestive system tumors (gastric cancer, intestinal cancer, pancreatic cancer, etc.) and chronic liver diseases (hepatitis B, liver fibrosis, liver cirrhosis, etc.) is improved.

5. The sanjie tablet can trans-differentiate hepatoma cells into normal liver cells. Some patients get the chance of radical operations because the size of the tumor body is obviously reduced, and some patients have conditions that are obviously improved, which are realized as pain relief or disappearance in the liver area, increased food intake and relief of abdominal distension, thereby prolonging the survival duration and improving the survival rate.

### DESCRIPTION OF THE EMBODIMENTS

Provided is a method for preparing an anti-hepatoma drug sanjie tablet, which includes a sanjie tablet containing components of Typhonii Rhizoma, exocarp of Juglans mandshurica, Dictamni Cortex and Bovis Calculus Artifactus. The preparation method includes the steps of:

step 1. removing toxins from the components of Typhonii Rhizoma, exocarp of Juglans mandshurica, and Dictamni Cortex in the sanjie tablet to obtain powder of nontoxic Typhonii Rhizoma, nontoxic exocarp of Juglans mandshurica, and nontoxic Dictamni Cortex; and

step 2. uniformly mixing the powder of nontoxic Typhonii Rhizoma, nontoxic exocarp of Juglans mandshurica, and nontoxic Dictamni Cortex obtained in step 1 with Bovis Calculus Artifactus, granulating with a volatile medium, drying, tabletting, and conducting film-coating to obtain the sanjie tablet.

Further, the removing toxins from the exocarp of Juglans mandshurica component in the sanjie tablet includes the steps of:

step 1. directly preparing fresh exocarp of Juglans mandshurica into a homogenate of exocarp of Juglans mandshurica, or drying and crushing fresh exocarp of Juglans mandshurica into coarse powder of exocarp of Juglans mandshurica;

step 2. taking probiotics and evenly mixing the same with distilled water at 20-40° C. according to the mass ratio of 1:5-50 to prepare a probiotic solution;

step 3. weighing 1 kg of the homogenate or coarse powder of exocarp of Juglans mandshurica from step 1, weighing 2-10 L of the probiotic solution from step 2, and mixing 1 kg of the homogenate or coarse powder of exocarp of Juglans mandshurica with 2-10 L of the probiotic solution according to a certain ratio to obtain a mixed raw stock slurry;

step 4. fermenting and culturing the mixed raw stock slurry obtained in the step 3 at a fermentable conversion temperature for 1 cycle, so as to conduct fermentable conversion with a ventilation ratio of 0-10 v/v and a stirring rate of 0-300 r/min to obtain a fermentation mixture; and

step 5. freeze-drying or oven-drying the fermentation mixture obtained in step 4 at a temperature of 40-80° C., and then crushing into fine powder as the nontoxic powder of exocarp of Juglans mandshurica.

Further, the probiotics is any one of probiotic Bacillus, clostridium butyicum, lactobacillus, bifidobacterium, saccharomycetes, medicated leaven and red yeast rice, or is at least prepared from any two or more of probiotic Bacillus, clostridium butyicum, lactobacillus, bifidobacterium, saccharomycetes, medicated leaven and red yeast rice.

Further, the OD600 of the probiotic solution >0.4.

Further, the fermentable conversion temperature is 25-45° C., and 1 fermentation cycle is 1-14 days.

Further, the removing toxins from the Dictamni Cortex component in the sanjie tablet includes the steps of:

step 1. crushing Dictamni Cortex into coarse powder of Dictamni Cortex;

step 2. taking probiotics and evenly mixing the same with distilled water at 20-40° C. according to the mass ratio of 1:5-50 to prepare a probiotic solution;

step 3. weighing 1 kg of the coarse powder of Dictamni Cortex from step 1, weighing 2-10 L of the probiotic solution from step 2, and mixing 1 kg of the coarse powder of Dictamni Cortex with 2-10 L of the probiotic solution according to a certain ratio to obtain a mixed raw stock slurry;

step 4. fermenting and culturing the mixed raw stock slurry obtained in the step 3 at a fermentable conversion temperature for 1 cycle, so as to conduct fermentable conversion with a ventilation ratio of 0-10 v/v and a stirring rate of 0-300 r/min to obtain a fermentation mixture; and

step 5. freeze-drying or oven-drying the fermentation mixture obtained in step 4 at a temperature of 40-80° C., and then crushing into fine powder as the nontoxic powder of Dictamni Cortex.

Further, the probiotics is any one of probiotic Bacillus, Clostridium butyicum, lactobacillus, bifidobacterium, saccharomycetes, medicated leaven and red yeast rice, or is at least prepared from any two or more of probiotic Bacillus, Clostridium butyicum, lactobacillus, bifidobacterium, saccharomycetes, medicated leaven and red yeast rice.

Further, the OD600 of the probiotic solution >0.4.

The removing toxins from the Typhonii Rhizoma component in the sanjie tablet includes the steps of:

step 1. crushing crude Typhonii Rhizoma into coarse powder;

step 2. taking probiotics and evenly mixing the same with distilled water at 20-40° C. according to the mass ratio of 1:5-50 to prepare a probiotic solution;

step 3. weighing 1 kg of crude Typhonii Rhizoma from step 2 and crushing into coarse powder, weighing 2-10 L of the probiotic solution from step 2, and mixing 1 kg of the coarse powder of crude Typhonii Rhizoma with 2-10 L of the probiotic solution according to a certain ratio to obtain a mixed proportional slurry;

step 4. fermenting and culturing the mixed proportional raw stock slurry obtained in step 3 at a fermentable conversion temperature for 1 cycle, so as to conduct fermentable conversion with a ventilation ratio of 0-10 v/v and a stirring rate of 0-300 r/min to obtain a proportional fermentation mixture; and

step 5. freeze-drying or oven-drying the proportional fermentation mixture obtained in step 4 at a temperature of 40-80° C., and then crushing into fine powder as the nontoxic powder of Typhonii Rhizoma.

Further, the probiotics is any one of probiotic Bacillus, Clostridium butyicum, lactobacillus, bifidobacterium, saccharomycetes, medicated leaven and red yeast rice, or is at least prepared from any two or more of probiotic Bacillus, Clostridium butyicum, lactobacillus, bifidobacterium, saccharomycetes, medicated leaven and red yeast rice.

Further, the OD600 of the probiotic solution >0.4.

Further, the fermentable conversion temperature is 25-45° C., and 1 fermentation cycle is 1-14 days.

Further, the volatile medium is ethanol.

### Example 1

Powder of nontoxic exocarp of Juglans mandshurica was prepared by the following method: fresh exocarp of Juglans mandshurica was directly homogenized; according to the volume ratio of 1:40, bifidobacterium and distilled water at 35° C. were taken and mixed evenly to prepare a bifidobacterium solution with OD600=0.63; then, according to the ratio of adding 2,800 mL of the bifidobacterium solution per 1 kg of the homogenate of exocarp of Juglans mandshurica, the bifidobacterium solution was taken and added into the homogenate of exocarp of Juglans mandshurica, mixed evenly, and fermented and cultured at the temperature of 30° C. for 4 days with the ventilation ratio of 2 (v/v) and the stirring rate of 150 r/min; and then the fermented mixture of exocarp of Juglans mandshurica after fermentation was oven-dried at the temperature of 45° C. and crushed into fine powder as the powder of nontoxic exocarp of Juglans mandshurica fermented by bifidobacterium.

### Example 2

Toxins were removed from the exocarp of Juglans mandshurica component by the following method: the dry medicinal material of exocarp of Juglans mandshurica was crushed into coarse powder; according to the volume ratio of 1:20, Saccharomyces cerevisiae and distilled water at 30° C. were taken and mixed evenly to prepare a Saccharomyces cerevisiae solution with OD600=0.81; then, according to the ratio of adding 3,000 mL of the Saccharomyces cerevisiae solution per 1 kg of the coarse powder of exocarp of Juglans mandshurica, the Saccharomyces cerevisiae solution was taken and added into the coarse powder of exocarp of Juglans mandshurica, mixed evenly, and fermented and cultured at the temperature of 36° C. for 3 days with the ventilation ratio of 5 (v/v) and the stirring rate of 200 r/min; and then the fermented mixture of exocarp of Juglans mandshurica after fermentation was oven-dried at the temperature of 55° C. and crushed into fine powder as the powder of nontoxic exocarp of Juglans mandshurica fermented by Saccharomyces cerevisiae.

### Example 3

Acute toxicity experiment of exocarp of Juglans mandshurica powder fermented by bifidobacterium:

210 Kunming mice (of SPF grade, weighed 18-22 g, half male and half female) were selected and randomly divided into 21 groups with 10 mice per group. The mice were fasted for 12 hours before the experiment, and had free access to water. Groups each administrated with exocarp of Juglans mandshurica powder fermented by bifidobacterium and fresh exocarp of Juglans mandshurica powder by gavage were respectively set with 10 concentration gradients and the gavage volume of 20 ml/kg body weight. The blank control group was given the same volume of an aqueous solution containing 3% Tween. The exocarp of Juglans mandshurica powder fermented by bifidobacterium was administrated three times a day, and the fresh exocarp of Juglans mandshurica powder was administrated once a day. When observation was conducted immediately after oral administration, it could be seen that the liveliness of mice in each group administrated with fresh exocarp of Juglans mandshurica was decreased with the increase of the administration close, with uncoordinated activities, stumbling on foot, squinting, glassy eyes, drowsiness, and being incapable of intaking food or water. After administration, the mice in low dose group had decreased motility, and the phenomena of piloerection and arching back, and the severity of the phenomena was directly proportional to the administration close. The mice in the group administrated with high dose were tired and lying at the bottom of the rearing cage, and were short of breath. 25 min after administration, some mice suffered from convulsions, spasms, drowsiness and immobility, disappearance of righting reflex could be observed 30 min after administration, and animals started to die 1 h after administration, with the death rate being directly proportional to the administration close. All mice died within 24 hours after gavage. After 14 days of continuous observation, all of the diet, appearance and behavior of the surviving mice were returned to normal from the next day, and no abnormal secreta and excreta were found. No symptoms of poisoning and animal death were found after 24 h. The LD50 of the fresh exocarp of Juglans mandshurica=23.52 g/kg. The LD50 of the powder of exocarp of Juglans mandshurica fermented by bifidobacterium >120 g/kg, and no obvious toxic reaction was found.

### Example 4

Toxins were removed from the Typhonii Rhizoma component by the following method:

The raw Typhonii Rhizoma was crushed into coarse powder; according to the volume ratio of 1:25, Lactobacillus plantarum and distilled water at 35° C. were taken and mixed uniformly to prepare a Lactobacillus plantarum solution with OD600=0.45; then, according to adding 4,500 mL of the Lactobacillus plantarum solution per 1 kg of the raw Typhonii Rhizoma powder, the Lactobacillus plantarum solution was taken and added into the coarse powder of raw Typhonii Rhizoma, mixed evenly, and fermented and cultured at the temperature of 28° C. for 6 days; and then the fermented mixture of Typhonii Rhizoma after fermentation was oven-dried at the temperature of 75° C. and crushed into fine powder as the powder of Typhonii Rhizoma fermented by Lactobacillus plantarum.

### Example 5

Acute toxicity experiment of Typhonii Rhizoma powder fermented by Lactobacillus plantarum:

210 Kunming mice (of SPF grade, weighed 18-22 g, half male and half female) were selected and randomly divided into 21 groups with 10 mice per group. The mice were fasted for 12 hours before the experiment, and had free access to water. Groups each administrated with Typhonii Rhizoma powder fermented by Lactobacillus plantarum and raw Typhonii Rhizoma powder by gavage were respectively set with 10 concentration gradients and the gavage volume of 20 ml/kg body weight. The blank control group was given the same volume of an aqueous solution containing 3%0 Tween. The Typhonii Rhizoma powder fermented by Lactobacillus plantarum was administrated three times a day, and the raw Typhonii Rhizoma powder was administrated once a day. Observation was conducted immediately after oral administration, the death of mice in each group within 24 hours was recorded, and the observation was conducted continuously for 14 days. The LD50 of raw Typhonii Rhizoma=5.7 g/kg. The LD50 of the powder of Typhonii Rhizoma fermented by Lactobacillus plantarum >120 g/kg, and no obvious toxic reaction was found.

### Example 6

The obtained powder of nontoxic Typhonii Rhizoma, nontoxic exocarp of Juglans mandshurica, and nontoxic Dictamni Cortex were evenly mixed with Bovis Calculus Artifactus, granulated with a volatile medium, dried, tabletted and coated with a film coating. Clinical research was conducted on recruited cases, wherein 201 cases of patients with middle and advanced stages of hepatic carcinoma who had lost the right time for surgery, chemotherapy, chemoradiotherapy, and the like were included, the sanjie tablet was orally administrated to the subjects with 6-8 tablets/time and 3 times/day, one month was regarded as a course of treatment, and each subject was subjected to no less than 3 courses of treatment. The therapeutic effect was observed through self control before and after the treatment, and administration of other anticancer drugs was stopped in each case during the medication period. All subjects were dynamically observed for the improvement of clinical symptoms, the changes of an intrahepatic tumor, and the changes of an alpha fetoprotein index before and after medication. The clinical effects on the recruited cases were shown in Table 1.

TABLE 1
Clinical data of oral administration of the sanjie
tablet in 201 patients with hepatoma

| | Determination | | |
|---|---|---|---|
| | of therapeutic | Number | Percentage |
| Mass change | effect | of cases | (%) |
| CR (with tumor | Complete | 13 | 6.5 |
| disappeared) | remission | | |
| PR (reduced by | Partial | 47 | 23.4 |
| more than 50%) | remission | | |
| MR (reduced by | Mild remission | 58 | 28.9 |
| 25-50%) | | | |
| No change | No change | 80 | 39.8 |
| Deterioration | Deterioration | 3 | 1.5 |

Therefore it could be seen that, by orally administrating the sanjie tablet to the patients with complete remission, the sanjie tablet could trans-differentiate hepatoma cells into normal liver cells. Some patients got the chance of radical operations because the size of the tumor body was obviously reduced, and some patients had conditions that were obviously improved, which were realized as pain relief or disappearance in the liver area, increased food intake and relief of abdominal distension, thereby prolonging the survival duration and improving the survival rate. The elongation rate was 89.28%. (P<0.01).

Comparison of therapeutic effects between the group orally administrated with the sanjie tablet and the group treated with transcatheter arterial chemoembolization (TACE) alone was shown in Table 2 and Table 3.

TABLE 2
Effect of the sanjie tablet on the maximum
mass diameter and alpha fetoprotein (AFP)

| | Maximum mass | Reduction rate | AFP | Decrease |
|---|---|---|---|---|
| Time | diameter (cm) | (%) | (μg/L) | rate (%) |
| Before | 5.8 ± 6.1 | 32.76 | 2024 ± 1836 | 51.68 |
| treatment | | | | |
| After | 3.9 ± 1.8 | | 978 ± 824 | |
| treatment | | | | |

As shown in Table 2, after oral administration of the sanjie tablet, the decrease rate of the maximum diameter of the tumor focus in liver was 32.76%, and the decrease rate of AFP was 51.68%, such that the therapeutic effect before and after treatment was significant (P<0.05).

TABLE 3
Comparison of the maximum mass diameter and alpha
fetoprotein (AFP) between the group administrated with
the sanjie tablet and the control group after 3 months

| | Maximum mass | | | |
|---|---|---|---|---|
| | diameter | | AFP | |
| Time | (cm) | P | (μg/L) | P |
| Chemoemboliza- | 3.81 ± 2.9 | <0.05 | 1545 ± 1163 | <0.01 |
| tion | | | | |
| Sanjie tablet | 2.72 ± 2.4 | | 978 ± 824 | |

As shown in Table 3, after three courses of treatment, the decrease in the maximum tumor diameter in the group administrated with the sanjie tablet was significantly stronger than that of the control group (P<0.05), and the decrease in alpha fetoprote in the group administrated with the sanjie tablet was also significantly stronger than that of the control group (P<0.01).

In view of the above, after fermentation, the toxicities of Typhonii Rhizoma, exocarp of Juglans mandshurica and Dictamni Cortex themselves had been removed, thereby improving the anti-tumor pharmaceutical effect. These effective components achieved a multi-target effect by inhibiting oncogenes, activating tumor-suppressor genes and repairing growth regulation genes. Through the transformed effective components, these mechanisms which had an effect on tumors are regulated at cellular and molecular levels, thereby exerting the anti-tumor, anti-recurrence and anti-metastasis functions. Upon biotransformation, macromolecular substances were changed into micromolecule substances, and thus were absorbed and utilized by the human body, thereby improving the pharmaceutical effect by 4 to 28 times.

### Example 7

Toxins were removed from the components of Typhonii Rhizoma, exocarp of Juglans mandshurica, and Dictamni Cortex in the sanjie tablet to obtain powder of nontoxic Typhonii Rhizoma, nontoxic exocarp of Juglans mandshurica, and nontoxic Dictamni Cortex after toxin removal. The powder of nontoxic Typhonii Rhizoma, nontoxic exocarp of Juglans mandshurica, and nontoxic Dictamni Cortex was evenly mixed with Bovis Calculus Artifactus, granulated with a volatile medium, dried, tabletted, and coated with a film coating.

The sanjie tablet obtained according to the aforementioned preparation method was studied for drug safety. 300 subjects in the clinic were divided into a treatment group, a control group and a group administrated with the sanjie tablet, with 100 subjects in each group. wherein:

treatment group: subjected to administration of the sanjie tablet in combination with transcatheter arterial chemoembolization;

control group: subjected to the transcatheter arterial chemoembolization alone;

group administrated with the sanjie tablet: self control before and after the treatment;

The experimental results were shown in Table 4.

TABLE 4
Clinical experiment results of drug safety in 300 cases administrated with the sanjie tablet

| | | | | | Comparison of the group | |
|---|---|---|---|---|---|---|
| | Comparison between the | | Comparison of the | | administrated with the | |
| | treatment group and the | | treatment group before | | sanjie tablet before | |
| | control group after treatment | | and after treatment | | and after treatment | |
| White blood cell | U = 1.98 | P < 0.05 | U = 0 | P > 0.05 | U = 0.377 | P > 0.05 |
| Hemoglobin | U = 0.12 | P > 0.05 | U = 0.16 | P > 0.05 | U = 1.13 | P > 0.05 |
| Blood platelet | U = 0.24 | P > 0.05 | U = 0.12 | P > 0.05 | U = 0.389 | P > 0.05 |

| | |
|---|---|
| Urine routine | No significant difference in various comparisons before and after treatment |
| Liver function | No significant difference in various comparisons before and after treatment |
| Kidney function | No significant difference in various comparisons before and after treatment |
| Chest X-ray analysis | No significant difference in various comparisons before and after treatment |
| Other | No numbness and swelling of tongue and lip, burning throat |
| | and diarrhea conditions were found in the treatment group |
| | and the group administrated with the sanjie tablet |

Conclusion: 1. it was not found that the sanjie tablet had any side effect on myelosuppression; 2. it was not found that the sanjie tablet had any adverse side effect on liver and kidney functions; 3. no toxic reaction of toxic components in medicinal herbs resources of the composition of the sanjie tablet was found during verification.

Therefore it could be seen that, the sanjie tablet obtained according to the aforementioned preparation method could ensuring the drug safety while improving the pharmaceutical effect, thereby making the patients in the clinic have no side effect of medication.

The present disclosure is not limited to the precise structure described above and shown in the drawings, and various modifications and changes can be made without departing from the scope of the present disclosure. The scope of the present disclosure is limited only by the appended claims.

What is claimed is:

1. A method for preparing an anti-hepatoma drug sanjie tablet, which comprises a sanjie tablet containing components of Typhonii Rhizoma, exocarp of Juglans mandshurica, Dictamni Cortex and Bovis Calculus Artifactus, the preparation method comprising the steps of:
step 1. removing toxins from the components of Typhonii Rhizoma, exocarp of Juglans mandshurica, and Dictamni Cortex in the sanjie tablet to obtain powder of nontoxic Typhonii Rhizoma, nontoxic exocarp of Juglans mandshurica, and nontoxic Dictamni Cortex; and
step 2. uniformly mixing the powder of nontoxic Typhonii Rhizoma, nontoxic exocarp of Juglans mandshurica, and nontoxic Dictamni Cortex obtained in step 1 with Bovis Calculus Artifactus, granulating with a volatile medium, drying, tabletting, and conducting film-coating to obtain the sanjie tablet.

2. The method for preparing an anti-hepatoma drug sanjie tablet according to claim 1, wherein the removing toxins from the exocarp of Juglans mandshurica component in the sanjie tablet comprises the steps of:
step 1. directly preparing fresh exocarp of Juglans mandshurica into a homogenate of exocarp of Juglans mandshurica, or drying and crushing fresh exocarp of Juglans mandshurica into coarse powder of exocarp of Juglans mandshurica;
step 2. taking probiotics and evenly mixing the same with distilled water at 20-40° C. according to the mass ratio of 1:5-50 to prepare a probiotic solution;
step 3. weighing 1 kg of the homogenate or coarse powder of exocarp of Juglans mandshurica from step 1, weighing 2-10 L of the probiotic solution from step 2, and mixing 1 kg of the homogenate or coarse powder of exocarp of Juglans mandshurica with 2-10 L of the probiotic solution according to a certain ratio to obtain a mixed raw stock slurry;
step 4. fermenting and culturing the mixed raw stock slurry obtained in the step 3 at a fermentable conversion temperature for 1 cycle, so as to conduct fermentable conversion with a ventilation ratio of 0-10 v/v and a stirring rate of 0-300 r/min to obtain a fermentation mixture; and
step 5. freeze-drying or oven-drying the fermentation mixture obtained in step 4 at a temperature of 40-80° C., and then crushing into fine powder as the nontoxic powder of exocarp of Juglans mandshurica.

3. The method for preparing an anti-hepatoma drug sanjie tablet according to claim 2, wherein the probiotics is any one of probiotic Bacillus, Clostridium butyicum, lactobacillus, bifidobacterium, saccharomycetes, medicated leaven and red yeast rice, or is at least prepared from any two or more of probiotic Bacillus, Clostridium butyicum, lactobacillus, bifidobacterium, saccharomycetes, medicated leaven and red yeast rice.

4. The method for preparing an anti-hepatoma drug sanjie tablet according to claim 2, wherein the OD600 of the probiotic solution >0.4.

5. The method for preparing an anti-hepatoma drug sanjie tablet according to claim 2, wherein the fermentable conversion temperature is 25-45° C., and 1 fermentation cycle is 1-14 days.

6. The method for preparing an anti-hepatoma drug sanjie tablet according to claim 1, wherein the removing toxins from the Typhonii Rhizoma component in the sanjie tablet comprises the steps of:
step 1. crushing crude Typhonii Rhizoma into coarse powder;
step 2. taking probiotics and evenly mixing the same with distilled water at 20-40° C. according to the mass ratio of 1:5-50 to prepare a probiotic solution;
step 3. weighing 1 kg of the coarse powder of crude Typhonii Rhizoma from step 1, weighing 2-10 L of the probiotic solution from step 2, and mixing 1 kg of the coarse powder of crude Typhonii Rhizoma with 2-10 L of the probiotic solution according to a certain ratio to obtain a mixed proportional slurry;
step 4. fermenting and culturing the mixed proportional slurry obtained in step 3 at a fermentable conversion temperature for 1 cycle, so as to conduct fermentable conversion with a ventilation ratio of 0-10 v/v and a stirring rate of 0-300 r/min to obtain a proportional fermentation mixture; and
step 5. freeze-drying or oven-drying the proportional fermentation mixture obtained in step 4 at a temperature of 40-80° C., and then crushing into fine powder as the nontoxic powder of Typhonii Rhizoma.

7. The method for preparing an anti-hepatoma drug sanjie tablet according to claim 6, wherein the probiotics is any one of probiotic Bacillus, Clostridium butyicum, lactobacillus, bifidobacterium, saccharomycetes, medicated leaven and red yeast rice, or is at least prepared from any two or more of probiotic Bacillus, Clostridium butyicum, lactobacillus, bifidobacterium, saccharomycetes, medicated leaven and red yeast rice.

8. The method for preparing an anti-hepatoma drug sanjie tablet according to claim 6, wherein the OD600 of the probiotic solution >0.4.

9. The method for preparing an anti-hepatoma drug sanjie tablet according to claim 6, wherein the fermentable conversion temperature is 25-45° C., and 1 fermentation cycle is 1-14 days.

10. The method for preparing an anti-hepatoma drug sanjie tablet according to claim 1, wherein the removing toxins from the Dictamni Cortex component in the sanjie tablet comprises the steps of:
step 1. crushing Dictamni Cortex into coarse powder of Dictamni Cortex;
step 2. taking probiotics and evenly mixing the same with distilled water at 20-40° C. according to the mass ratio of 1:5-50 to prepare a probiotic solution;
step 3. weighing 1 kg of the coarse powder of Dictamni Cortex from step 1, weighing 2-10 L of the probiotic solution from step 2, and mixing 1 kg of the coarse powder of Dictamni Cortex with 2-10 L of the probiotic solution according to a certain ratio to obtain a mixed raw stock slurry;
step 4. fermenting and culturing the mixed raw stock slurry obtained in the step 3 at a fermentable conversion temperature for 1 cycle, so as to conduct fermentable conversion with a ventilation ratio of 0-10 v/v and a stirring rate of 0-300 r/min to obtain a fermentation mixture; and
step 5. freeze-drying or oven-drying the fermentation mixture obtained in step 4 at a temperature of 40-80° C., and then crushing into fine powder as the nontoxic powder of Dictamni Cortex.

11. The method for preparing an anti-hepatoma drug sanjie tablet according to claim 10, wherein the probiotics is any one of probiotic Bacillus, Clostridium butyicum, lactobacillus, bifidobacterium, saccharomycetes, medicated leaven and red yeast rice, or is at least prepared from any two or more of probiotic Bacillus, Clostridium butyicum, lactobacillus, bifidobacterium, saccharomycetes, medicated leaven and red yeast rice.

12. The method for preparing an anti-hepatoma drug sanjie tablet according to claim 10, wherein the OD600 of the probiotic solution >0.4.

13. The method for preparing an anti-hepatoma drug sanjie tablet according to claim 10, wherein the fermentable conversion temperature is 25-45° C., and 1 fermentation cycle is 1-14 days.

14. The method for preparing an anti-hepatoma drug sanjie tablet according to claim 1, wherein the volatile medium is ethanol.
